(19) United States
(12) Patent Application Publication
(10) Pub. No.: US 2013/0245727 A1
(43) Pub. Date: Sep. 19, 2013

(54) SYSTEMS AND METHODS FOR THERMOLIPOLYSIS USING RF ENERGY

(71) Applicant: CUTERA, INC., Brisbane, CA (US)

(72) Inventors: Amogh KOTHARE, Fremont, CA (US); Leonard C. DEBENEDICTIS, Brisbane, CA (US); Dragan NEBRIGIC, Livermore, CA (US)

(73) Assignee: CUTERA, INC., Brisbane, CA (US)

(21) Appl. No.: 13/799,424

(22) Filed: Mar. 13, 2013

Related U.S. Application Data
(60) Provisional application No. 61/612,092, filed on Mar. 16, 2012.

(51) Int. Cl. A61N 1/32

(52) U.S. Cl. CPC A61N 1/328
USPC 607/102; 607/101

(57) ABSTRACT

Disclosed herein are systems and methods for preferentially heating subcutaneous tissue. Systems for heating subcutaneous tissue may comprise one or more radio-frequency (RF) electrodes having contoured tissue contacting surfaces, such as electrodes with concave or convex surface contours. Methods may comprise applying RF voltage or current from one or both of the handpieces to attain a target tissue and/or electrode temperature and to maintain the tissue and/or electrode at that temperature.

### CROSS-REFERENCE TO RELATED APPLICATIONS

This application claims priority to U.S. Provisional Patent Application Ser. No. 61/612,092, filed Mar. 16, 2012, which is hereby incorporated by reference in its entirety.

### BACKGROUND

Various forms of electrosurgery are widely used for a vast range of surgical procedures. For example, electrosurgery may be used for non-invasive interventions for subcutaneous fat reduction or diminution of the appearance of cellulite. Some cosmetic skin treatments effect dermal heating by applying radiofrequency (RF) energy to the skin using surface electrodes. The local heating is intended to tighten the skin by producing thermal injury that changes the ultrastructure of collagen in the dermis, and/or results in a biological response that changes the dermal mechanical properties. However, despite the efficacy of applying RF energy for skin tightening, it can be seen that there is a need for an electrosurgical system that decreases the risk of patient burns and discomfort. There is a further need for an effective modality by which subcutaneous fat tissue may be non-invasively reshaped, and/or removed for improving the appearance of human skin or for sculpting the human body, without heating non-targeted body structures.

### BRIEF SUMMARY

Disclosed herein are systems and methods for preferentially heating subcutaneous tissue. In some variations, these systems and methods may be used for heating adipose cells that are located below the surface of the skin. Systems for preferentially heating subcutaneous tissue may comprise one or more RF electrodes having contoured tissue contacting surfaces, such as electrodes with rounded or domed surface contours. In some variations, such systems may also comprise a vacuum source that may be activated periodically and/or in conjunction with the application of RF energy from the surface-contoured electrodes for heating subcutaneous tissue. Systems for heating subcutaneous tissue may also comprise two handpieces, each handpiece having one or more surface-contoured RF electrodes. Methods may comprise applying RF voltage or current from one or both of the handpieces. In some variations, there may be a phase difference between the RF voltages or currents of the two handpieces. Alternatively or additionally, vacuum may be provided in concert with RF energy. Intermittent or pulsatile application of vacuum to a tissue region may help to increase blood perfusion to that tissue region. Such synergistic interaction between the application of RF energy from one or both handpieces and/or the application of vacuum to a tissue region may help to preferentially heat targeted subcutaneous tissue (such as adipose tissue) without excessively heating non-targeted adjacent tissue (such as superficial surface tissue, skin or muscle tissue).

One variation of a system for heating subcutaneous tissue may comprise a first handpiece having a first surface-contoured electrode and a first vacuum source and a controller configured to activate the first vacuum source when the first surface-contoured electrode is not activated. The system may further comprise a second handpiece having a second surface-contoured electrode and a second vacuum source, where the controller may be configured to activate the second vacuum source when the second surface-contoured electrode is not activated. The first surface-contoured electrode may comprise an outer planar portion along the perimeter of the electrode and an inner domed portion enclosed by the outer planar portion. The inner domed portion of the electrode may be hollow and the outer planar portion may be solid.

One variation of a method for heating subcutaneous tissue may comprise contacting a patient's skin with a first handpiece having a first domed electrode and a first vacuum source, contacting a patient's skin with a second handpiece having a second domed electrode and a second vacuum source, activating the first handpiece such that the first domed electrode applies RF energy to the skin at a first frequency and the first vacuum source is activated out-of-phase from the first electrode, and activating the second handpiece such that the second domed electrode applies RF energy to the skin at a second frequency and the second vacuum source is activated out-of-phase from the second electrode. In some variations, the first and second frequencies are the same, and may be in phase with each other or may have a non-zero phase shift. The first handpiece and the second handpiece may be activated at substantially at the same time or may be activated with a phase difference between them.

Another variation of a system for heating subcutaneous tissue may comprise a handpiece having an electrode and a controller configured to apply a voltage to the electrode to apply RF energy to tissue. The electrode may have a tissue-contacting surface and a coating disposed over the tissue-contacting surface. The coating may comprise a dielectric material and have a thickness from about 3 micrometers to about 100 micrometers. In some variations, the coating may be polyurethane. The tissue-contacting surface of the electrode may be convex. The system may also comprise a temperature sensor that is located within the electrode and in contact with the tissue-contacting surface.

Another variation of a system for heating subcutaneous tissue may comprise a handpiece having an electrode, a temperature sensor in contact with the electrode, and a controller coupled to the temperature sensor and configured to apply a voltage to the electrode to apply RF energy to the tissue. The electrode may have a tissue-contacting surface and a dielectric film disposed over the tissue-contacting surface. The temperature sensor may be in contact with the tissue-contacting surface of the electrode. The controller may be programmed to apply a first voltage level during a first phase while the tissue temperature is being increased and during a second phase following the first phase, the voltage may be adjusted to maintain a target temperature of the tissue. During the second phase, the voltage level may be restricted not to exceed a second voltage level, where the second voltage level less than the first voltage level. Optionally during the first phase, the first voltage level may be maintained until the target temperature is reached. Alternatively, the first voltage level may be maintained during the first phase until a stop temperature of the tissue is reached, where the stop temperature may be below the target temperature.

One variation of a device for heating subcutaneous tissue may comprise a handpiece having an electrode, where the electrode has a tissue-contacting surface and a dielectric film disposed over the tissue-contacting surface. The film may be uniformly stretched over the tissue-contacting surface. In some variations, the device may comprise two dielectric films where the first dielectric film is disposed over the tissue-contacting surface of the electrode and the second dielectric film is uniformly stretched over the tissue-contacting surface and on top of the first dielectric film. The total thickness of the first and second films may be 50 micrometers. In some variations, each of the one or more films may have a thickness from about 10 micrometers to about 100 micrometers.

Each of the first and second films may be stretched over the tissue-contacting surface by attaching the film to a frame, applying an adhesive to one side of the film, and applying the frame to the electrode at an angle such that the side of the film with the adhesive contacts the electrode without any air pockets between the tissue-contacting surface and the film. The adhesive may be an acetate adhesive and the one or more films may be polyurethane films. In some variations, the film may have an electrical conductivity range of about 1×10⁻⁸ S/m to about 100×10⁻⁸ S/m, and/or may have a permittivity range from about 1 to about 7. In some variations, the tissue-contacting surface of the electrode may have a radius of curvature from about −400 mm to about +400 mm, and may be concave or convex. In other variations, the tissue-contacting surface may be flat.

### BRIEF DESCRIPTION OF THE DRAWINGS

FIG. 1 depicts one variation of a control console for a RF system for heating subcutaneous tissue.

FIGS. 2A-2C depict perspective, side, and bottom elevational views of one variation of a handpiece that may be used with a RF system for heating subcutaneous tissue.

FIGS. 3A-3C depict bottom, cross-sectional, and side view of a variation of an RF electrode.

FIGS. 4A and 4B depict perspective and cross-sectional views of the internal components of different variations of a handpiece.

FIGS. 5A to 5C depict schematic representations of various methods of using a RF system for heating subcutaneous tissue.

FIGS. 6A and 6B depict examples of an experiment to determine temperatures at which cell viability is disrupted.

FIGS. 7A to 7D depict an example of an experiment to determine temperature distributions attained by applying RF energy using a dome electrode.

FIGS. 8A to 8D depict an example of an experiment to show changes in macrophage activity due to heat treatment.

FIGS. 9A-9F depict an example of an experiment to evaluate pressure-induced vasodilation.

FIGS. 10A and 10B depict perspective and side views of another variation of a handpiece that may be used with a RF system for heating subcutaneous tissue. FIG. 10C is a cross-sectional perspective view of the handpiece of FIGS. 10A and 10B. FIG. 10D is a perspective view of a printed circuit board assembly inside the handpiece.

FIGS. 11A-11D depict bottom, top, cross-sectional and side views of a variation of a RF electrode.

FIG. 12A depicts the current and voltage waveforms applied to the electrode to attain and maintain a target temperature of the electrode and/or tissue. FIG. 12B depicts another variation of current and voltage waveforms that may be applied to the electrode and/or tissue.

### DETAILED DESCRIPTION

The systems and methods for heating subcutaneous tissue disclosed herein may be used for body sculpting or contouring. For example, such systems and methods may be used for deep tissue heating and/or the temporary reduction in the appearance of cellulite. In one variation, a system may comprise one or more handpieces each comprising one or more electrodes for applying RF energy configured to provide large volumetric deep tissue treatment. For example, a system for heating subcutaneous adipose fat may comprise dual handpieces (e.g., two handpieces) each having at least one electrode and one or more temperature sensors associated with the electrode, and a controller that configured to apply RF energy using real-time temperature feedback. This may allow a practitioner to control the depth and degree of tissue heating across a wide range of treatment areas, and may help ensure that subcutaneous tissue is preferentially heated over surface tissue. Examples of treatment areas may include, but are not limited to, the abdomen, flanks, thighs, buttocks. While the examples below describe the system and methods in the context of heating subcutaneous fat tissue, it should be understood that these systems and methods may be used to preferentially heat any tissue structure, as may be desirable.

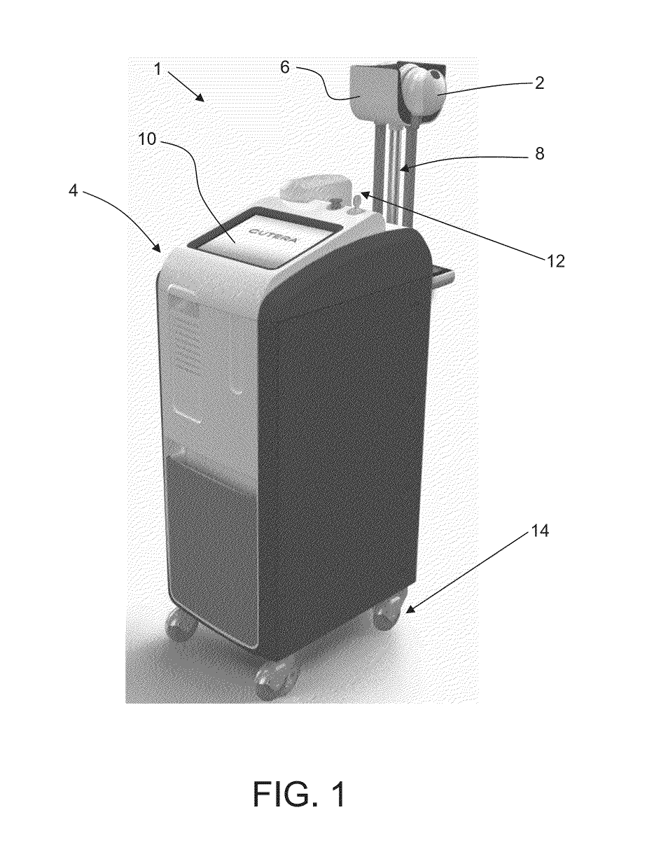

FIG. 1 depicts one variation of a system that may be used for preferentially heating subcutaneous adipose tissue. As illustrated there, RF system 1 may comprise a handpiece 2 and a control console 4. Optionally, RF system 1 may also comprise a second handpiece in addition to first handpiece 2. The handpiece 2 may comprise at least one electrode, such as the surface-contoured electrodes described below. RF system 1 may also comprise one or more ground pads that may be positioned around a patient's body with respect to the handpiece(s) (e.g., ground pad(s) may be positioned at non-target regions of the body). The handpiece 2 may be stored in a cradle 6 that is attached to the control console 4, and may comprise wires, tubes and cables 8 that connect it to the console 4. The wires, tubes and cables 8 may comprise electrical wires that convey current or voltage to the handpiece 2 for the application of RF energy, as well as control signals to control the operation of the handpiece. Optionally, the wires, tubes and cables 8 may also comprise various tubes, such as vacuum tubes and/or fluid tubes, to provide negative pressure and/or fluid (e.g., water) to the handpiece 2. The console 4 may comprise a vacuum source and/or fluid reservoir, as well as an electrosurgical generator to drive these functions in the handpiece 2. As depicted in FIG. 1, the console 4 may also comprise a display screen 10 and one or more use-activated controls 12 that may allow a practitioner to control the RF energy, vacuum, and/or fluid supplied to the handpiece 2, as well as to provide feedback from any system sensors (e.g., temperature sensors, skin contact sensors, and the like) to the practitioner. The console 4 may optionally comprise wheels 14 that allow the RF system 1 to be moved, as may be desirable.

The electrosurgical generator provided with the RF systems described herein may be configured to supply RF energy to a handpiece with a frequency of about 300 kHz to about 2 MHz, with a maximum power of about 150 W and/or maximum current of about 1.5 A. The electrosurgical generator may have at least two operating modes, where the first operating mode comprises applying a constant power to the electrodes and the second operating mode comprises applying a constant current to the electrodes. In a dual-handpiece RF system, there may be a single electrosurgical generator that drives both handpieces, or two electrosurgical generators to separately drive each handpiece. The controller for a dual-handpiece RF system may be configured to drive the two handpieces simultaneously (e.g., at the same or different frequency and/or power and/or current), and/or may be configured to drive the two handpieces alternately (e.g., with a non-zero phase shift). For example, the controller may be configured to drive the two handpieces with a phase shift between them from about 0° to about 180°. For example, the two handpieces may be operated in phase (e.g., phase shift of about 0°) in conjunction with a ground pad located remotely from the target tissue, which may act to heat tissue deep beneath the surface of the skin (e.g., current may flow from each of the two handpieces to the remotely located ground pad). The two handpieces may be operated out of phase (e.g., phase shift of about 180°), which may act to heat shallow tissues near the surface of the skin (e.g., current may flow along more superficial tissue between the two handpieces). Adjusting the phase shift may allow a practitioner to control the depth of the tissue that is to be heated.

### Handpiece

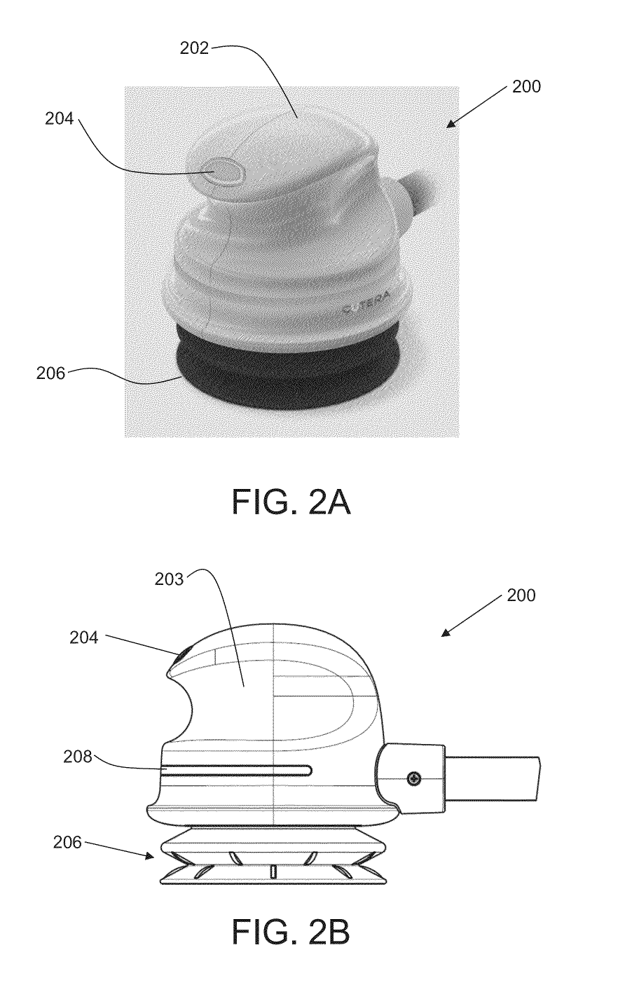

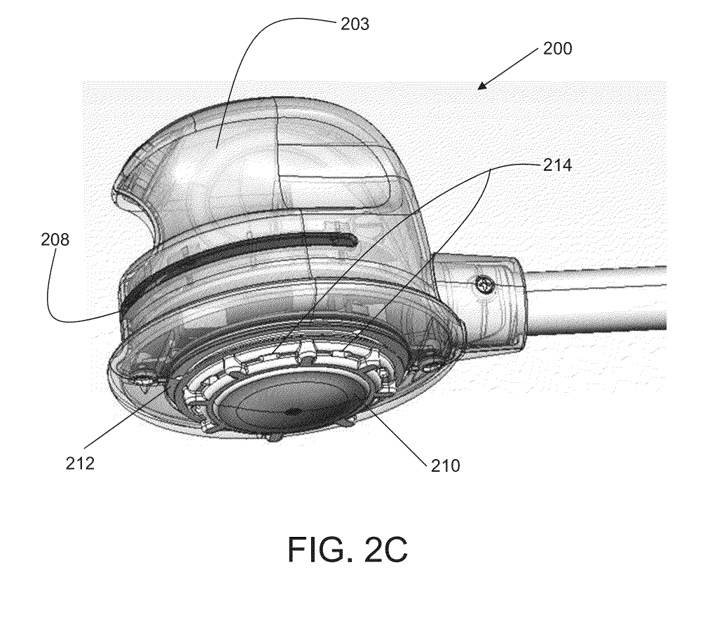

RF system 1 may comprise one or more handpieces, and in some variations, may be a dual-handpiece system comprising two handpieces. One example of a handpiece 200 is depicted in FIGS. 2A-2C. Handpiece 200 may comprise an ergonomic housing 202 and a control button 204. The handpiece 200 may optionally comprise flanges or bellows 206, which may be used to contact a patient's skin (e.g., for the application of vacuum suction). The control button 204 may be used to turn the handpiece 200 on or off. The ergonomic housing 202 may have a shape suitable for manual gripping so that a practitioner may contact the handpiece 200 to a target area on a patient (e.g., a groove or recessed region 203 may be sized and shaped for gripping with one or more fingers). As illustrated in FIG. 2B, the handpiece 200 may also comprise an indicator light 208, which may indicate the operational state of the handpiece (e.g., whether the handpiece is on or off, whether it is charged and ready to deliver RF energy, sleep mode, etc.). The indicator light 208 may be a strip that wraps around at least a portion of the housing 202, or may have any desired shape and size. In some examples, the indicator light 208 may change between two or more colors to indicate two or more operating modes of the handpiece. The indicator light 208 may be illuminated using one or more LEDs (e.g., one or more LEDs distributed along the illumination strip).

The bellows 206 may define a space or chamber around the skin where vacuum may be applied. One or more electrodes of the handpiece 200 may be located within the bellows chamber. FIG. 2C depicts handpiece 200 with the bellows 206 removed for clarity. The bellows 206 may be attached to handpiece 200 by a circular frame 212. The frame 212 may comprise a plurality of apertures 214 (e.g., distributed along the circumference of the frame 212) for the application of negative pressure to the bellows chamber. The frame 212 may circumscribe a surface-contoured electrode 210, which may allow RF energy to be applied to skin located within the bellows chamber. As will be described in greater detail below, RF energy and vacuum may be applied to a patient's skin simultaneously or alternately, as may be desirable. While the bellows, frame and electrode are depicted as generally circular, it should be understood that they may have any shape suitable for applying RF energy and/or vacuum to a target skin region. For example, the bellows, frame and electrode may be shaped as a rectangle, square, ellipse, or any shape (irregular or otherwise) that may be tailored for a particular body region.

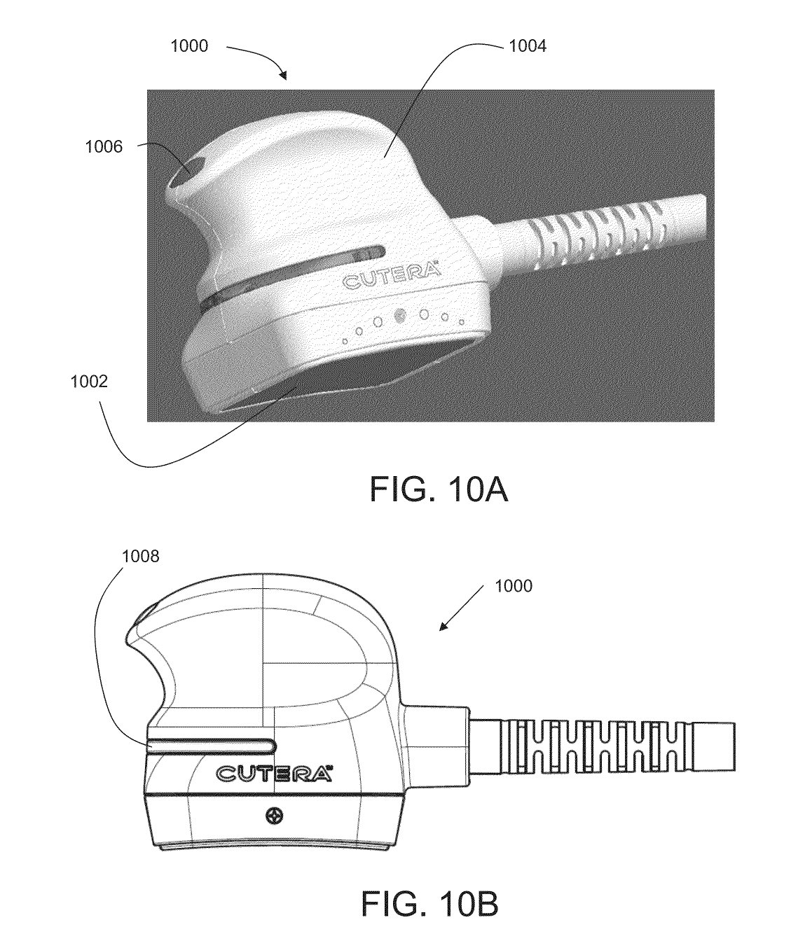

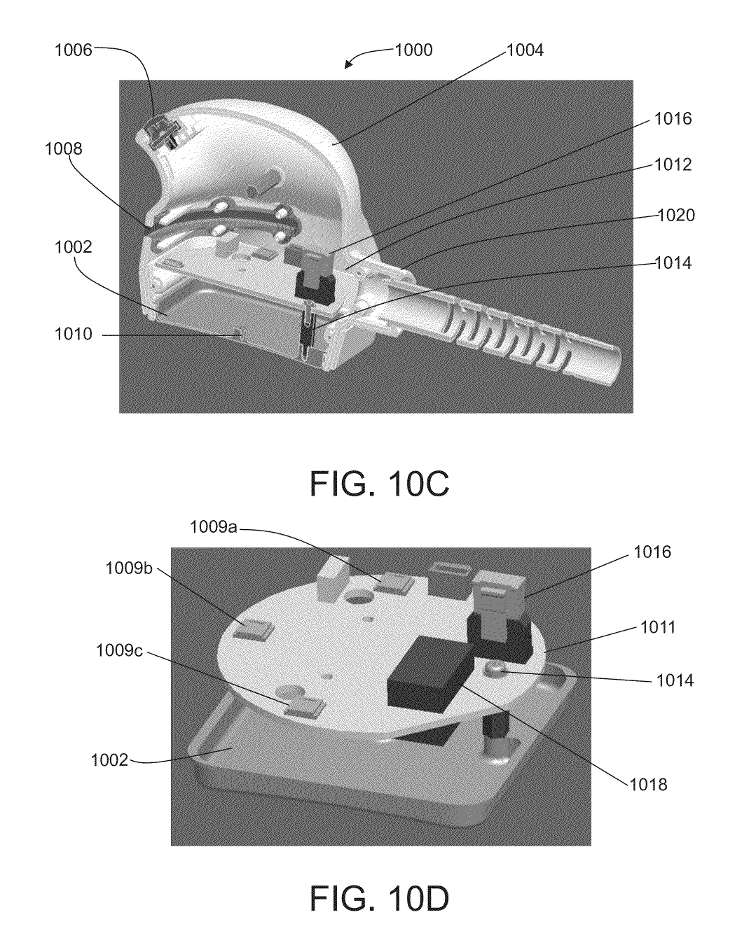

FIGS. 10A and 10B depict one variation of a handpiece 1000 with a square-shaped electrode 1002. The handpiece may comprise an ergonomic housing 1004 and a control button 1006. The ergonomic housing 1004 may have a shape suitable for manual gripping so that a practitioner may contact the handpiece 1000 to a target area on a patient (e.g., a groove or recessed region may be sized and shaped for gripping with one or more fingers). While the handpiece 200 may have bellows, the handpiece 1000 does not have bellows. Optionally, the handpiece 1000 may also comprise an illuminated indicator 1008 which may be configured to display lights of different colors and/or at different frequencies to indicate the operational state of the handpiece. For example, the illuminated indicator 1008 may display lighting that indicates an “RF ON” state, and/or “READY” state, and/or “STANDBY” state, and/or “ERROR” state. The electrode 1002 may have a concave surface contour, which may be useful when applying RF energy to rounded (e.g., convex) anatomical regions. Electrodes with various shapes, surface contours, and coatings or films are further described below.

FIGS. 10C and 10D depict one variation of a printed circuit board assembly (PCBA) 1012 that may be used in a RF handpiece, for example, the handpiece 1000 depicted in FIGS. 10A and 10B. The PCBA 1012 may comprise a printed circuit board 1011, RF energy source 1016, a noise filtering circuit 1018, one or more LEDs 1009a, 1009b, 1009c, and a conduit 1014 between the RF energy source 1016 and the electrode 1002. A port 1020 on the handpiece may connect with a bus from the system controller (not shown) to drive the various components on the PCBA, which may be connected to each other via interconnect wires in various layers of the printed circuit board 1011. In some variations, the conduit 1014 may be an electrically conductive screw that extends between the printed circuit board 1011 and the electrode 1002. The conduit 1014 may be connected to an output of the RF energy source 1016 via an inner layer of the printed circuit board 1011. The one or more LEDs 1009a, 1009b, 1009c may provide light to the illuminated indicator 1008, and each of the LEDs may be activated based on one or more signals from the system controller.

As depicted in FIG. 10C, the handpiece 1000 may also comprise a temperature sensor such as thermistor 1010 located in the center of the electrode 1002. The temperature sensed by the thermistor may be the temperature of the electrode, but in variations where the electrode is thermally conductive, the temperature of the electrode may be equivalent to the temperature of the skin that is in contact with the electrode. The thermistor 1010 may be in communication with the system controller, which may use the provided temperature data of the electrode and/or skin to modulate voltage output to the electrode, thereby modulating the level of RF energy delivered to the tissue. A handpiece may have one or more thermistors at one or more locations on the electrode to communicate temperature data to the controller to further refine the voltage output to the electrode. The noise filtering circuit 1018 may be provided to reduce interference between the RF energy source 1016 and the thermistor 1010, so that the generated RF energy does not interfere with the thermistor and give rise to erroneous temperature readings. In a preferred embodiment, the electrode should be at least 500 microns thick to facilitate heat dissipation.

### Electrode

A portion of a surface-contoured electrode may comprise a tissue-contacting surface that protrudes with a substantially convex geometry. Such a protrusion may help to provide homogeneous heating of tissue contacting the surface of the electrode. In some variations, a portion of the tissue-contacting surface may protrude with a shape that resembles a dome, and/or may comprise one or more curves which may be tapered, symmetric, asymmetric, etc. In other variations, portions of the tissue-contacting surface may have a pattern of protruding curves, and may have a corrugated pattern across the surface. A surface-contoured electrode may have one or more coatings that may prevent scratches as well as capacitive coupling between the electrode and the skin. Coatings may also help to prevent dielectric breakdown. Some variations of surface-contoured electrodes may be solid, and while other variations may have one or more hollow regions.

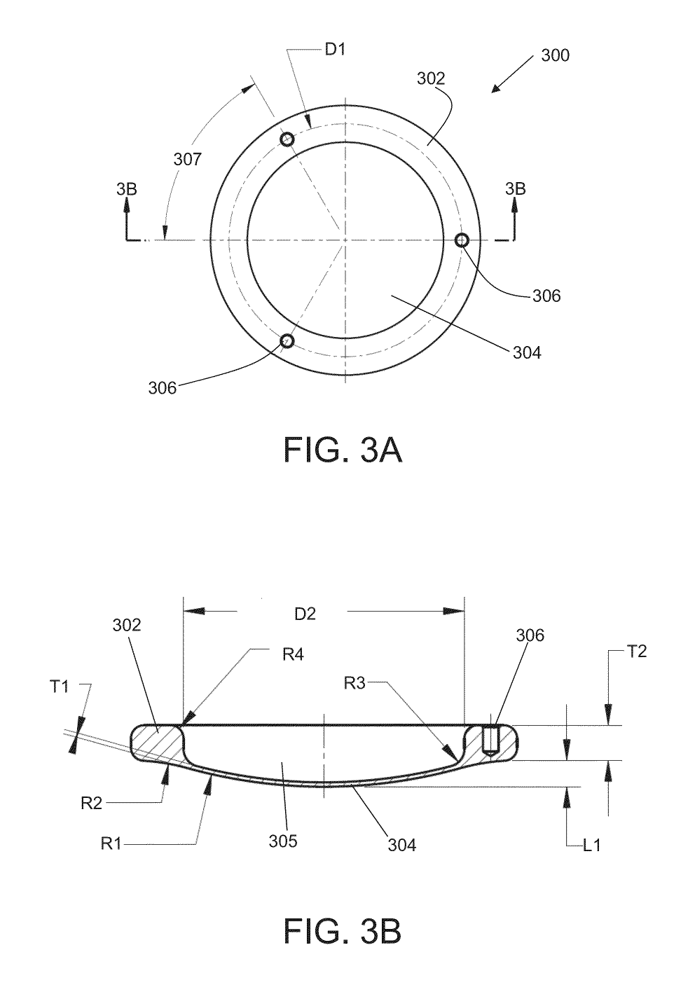

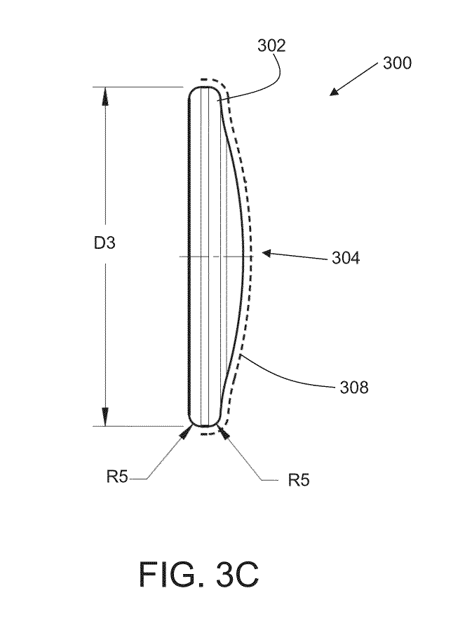

FIGS. 3A-3C depict one example of a dome-shaped surface-contoured electrode 300 that may be used with a RF system for heating subcutaneous adipose tissue. FIG. 3A depicts a front view of the tissue-contacting surface of the electrode 300. The electrode 300 may comprise an outer portion 302 that is substantially planar and flat that circumscribes an inner domed portion 304 that protrudes from the outer planar portion. The electrode 300 may also comprise one or more holes 306 that are spaced apart (e.g., within the outer planar portion 302) for attaching the electrode 300 to the handpiece. The angular spacing 307 between the holes 306 may vary between about 10° to about 180°, depending on the number of holes. For example, with three holes, the angular spacing 307 may be about 60°. The diameter D1 of the outer portion 302 may be from about 1 inch to about 2 inches, e.g., 1.496 inches. FIG. 3B depicts a cross-section of electrode 300 along the line 3B-3B. As shown there, the inner domed portion 304 may be hollow, while the outer planar portion 302 may be substantially solid (e.g., solid except for the holes 306). The diameter D2 of the inner domed portion 304 may be from about 0.75 inch to about 1.5 inches, e.g., about 1.26 inches. The thickness T1 of the inner domed portion 304 may be from about 0.005 inch to about 0.1 inch, e.g., about 0.02 inch. The length L1 with which the inner domed portion 304 extends from the outer planar portion 302 may be from about 0.05 inch to about 0.2 inch, e.g., about 0.118 inch. The thickness T2 of the outer planar region 302 may be from about 0.05 inch to about 0.2 inch, e.g., about 0.157 inch. The radius of curvature R1 of the inner domed portion 304 may be from about 1.5 inches to about 3 inches, e.g., about 2.29 inches. The radius of curvature R2 of the transitional region between the inner domed portion 304 and the outer portion 302 may be from about 0.5 inch to about 1.2 inches, e.g., about 0.945 inch. The interior edge of the inner domed portion 304 may have a radius of curvature R3 from about 0.04 inch to about 0.15 inch, e.g., about 0.079 inch. In some variations, the radius of curvature R3 may be from about −400 mm to about +400 mm. The interior edge of the outer planar portion 302 may have a radius of curvature R4 from about 0.01 inch to about 0.15 inch, e.g., about 0.059 inch. FIG. 3C depicts a side view of the surface-contoured electrode 300. The diameter D3 of the entire electrode 300 may be from about 0.5 inch to about 2.5 inches, e.g., about 1.732 inches. Both of the external edges of the outer planar portion 302 may have a radius of curvature of about 0.025 inch to about 0.12 inch, e.g., about 0.059 inch.

In some variations, one or more temperatures sensors (e.g., thermistors) may be located in cavity 305 of the inner domed portion 304. For example, there may be 2, 3, 4, 6, 10 or more thermistors located at the thinnest portion of the inner domed portion to measure the temperature of the skin directly contacting the electrode 300. In some variations, these temperature measurements may fed back to the system controller, which may then adjust the frequency and magnitude of the RF energy supplied to the electrode to ensure that the skin temperature remains within a certain range. For example, the system controller may be programmed to maintain the skin temperature between a minimum temperature and a maximum temperature, where such temperatures may be selected on a patient-by-patient basis, or may be selected depending on the body region that is treated. In some variations, the thermistors may be electrically isolated from the electrode to help prevent RF coupled noise from corrupting the temperature data. Alternatively or additionally, the temperature of the electrode and/or skin may be monitored by non-contact thermo-sensing methods, such as infrared (IR) thermography. One or more IR sensors may be located on a PCB of the handpiece facing the cavity 305 of the surface-contoured electrode. The portion of the electrode that is subject to IR exposure may be coated with a flat black finish, which may help to prevent erroneous temperature readings. For example, a circular region of the wall of the cavity 305 may be coated with a flat black finish, where the radius of the circular region may be from about 0.5 cm to about 1.0 cm.

Optionally, the electrode 300 may comprise one or more coatings or films 308, as schematically represented by the dotted line in FIG. 3C. Examples of coatings or films that may be used may include, but are not limited to, nickel, polytetrafluoroethylene (PTFE, e.g., Dupont Teflon® 420-104), fluorinated ethylene propylene (FEP), perfluoroalkoxy (PFA), Xylan®, polyvinyl fluoride, ACLAR, polyurethane, biocompatible polymers and plastics, anodized coatings and the like. The coating or film 308 may be applied to the entire skin-contacting surface of the electrode (e.g., both the outer planar portion and the inner domed portion), or just the surface-contoured portion of the electrode. The coating(s) or film(s) 308 may have a thickness from about 0.0001 inch to about 0.005 inch, e.g., about 0.00038 inch.

In one variation, an electrode may comprise one or more dielectric films disposed over the skin-contacting surface of the electrode. The dielectric strength of the film may be high enough to sustain electrode voltages of about 250 Vrms without breaking down. The film may also have a dielectric strength that can sustain electrode voltages up to 1500 V without breaking down. The film may have an electrical conductivity from about 1×10⁻⁸ S/m to about 100×10⁻⁸ S/m, e.g., 1×10⁻⁸ S/m. The film may also have a permittivity range from about 1 to about 7, e.g., 2. The film may also be made of a material that is resistant to the pinhole effect under the operating conditions of the handpiece. For example, the film may be able to maintain its integrity in the face of electrode currents in the range of about 0.2 Amps to about 1.5 Amps, electrode voltages in the range of about 10V to about 240 V, and electrode temperatures in the range of about 20C to about 55 C. In some variations, the film may comprise polyurethane, mylar, polyimide (e.g., Kapton®), and the like. The film may have a thickness between about 1 micrometer and about 100 micrometers, e.g., 3 micrometers, 10 micrometers, 25 micrometers, 50 micrometers, 75 micrometers, etc. One or more films may be applied over the skin-contacting surface of the electrode, where a first film is applied to the electrode and a second film is applied over the first film. For example, a first polyurethane film having a thickness of about 25 micrometers may be disposed over the electrode and a second polyurethane film having a thickness of about 25 micrometers may be disposed over the first film, for a total film thickness of about 50 micrometers. In still other variations, an electrode may comprise more than two films. The film(s) may be attached to the electrode by any suitable method. For example, the film may be stretched over the skin-contacting surface of the electrode and attached to the electrode by an adhesive, such as an acetate adhesive. The film may be applied uniformly over the electrode in such a way that avoids the formation of air pockets between the film and the electrode. One variation of a method for applying one or more films to the skin-contacting surface of an electrode is described below.

Various methods may be used to apply one or more films to an electrode such that the film is evenly distributed across the surface of the electrode and there are no air pockets or bubbles between the film and the electrode surface. One variation of a method may comprise stretching the film (e.g. a polyurethane film or sheet) across a frame such that the film is taut, applying an adhesive to one side of the film (e.g., an acetate adhesive), positioning the film at an angle with respect to the surface of the electrode, and rolling the film across the surface of the electrode such that the adhesive attaches the film to the electrode. The frame may have any suitable shape with a central open space across which the film may be stretched and temporarily secured while it is positioned with respect to the electrode. For example, the frame may be ring-shaped. The film may be positioned about 30-45 degrees with respect to the electrode surface before the film is rolled from one end of the electrode to the other. Sequentially contacting the film to the electrode surface from one edge to the other may help to ensure that no air pockets (e.g., bubbles) are trapped between the film and the electrode surface. A second film may be applied over the electrode and in contact with the first film in a similar manner. Even and uniform application of the one or more films over the electrode may help to ensure a uniform current density when the electrode is activated. Other methods that may be used to apply films and/or coatings to an electrode may include sputter deposition, physical vapor deposition, dip coating, etc.

In some variations, a handpiece may comprise one or more electrode contact sensors located near the electrode. The electrode contact sensors may provide an indicator to a practitioner of the degree of electrode-skin contact, and may help to provide an alarm to the practitioner is electrode-skin contact is lost. In some variations, the electrode contact sensors may detect a change in impedance, voltage, and/or current, where the change correlates to a level of electrode-skin contact. A controller may be programmed to cease the application of RF energy to an electrode that is not adequately contacted to a patient's skin. In other examples, the degree of electrode-skin contact may be determined by measuring changes in the load impedance of the electrode. For instance, an increase in electrode impedance may indicate a loss of electrode-skin contact (e.g., poor electrode-skin contact), while a decrease in electrode impedance may indicate stable electrode-skin contact (e.g., firm, full, and/or consistent electrode-skin contact, where substantially the entire conductive surface of the electrode is contacting the skin).

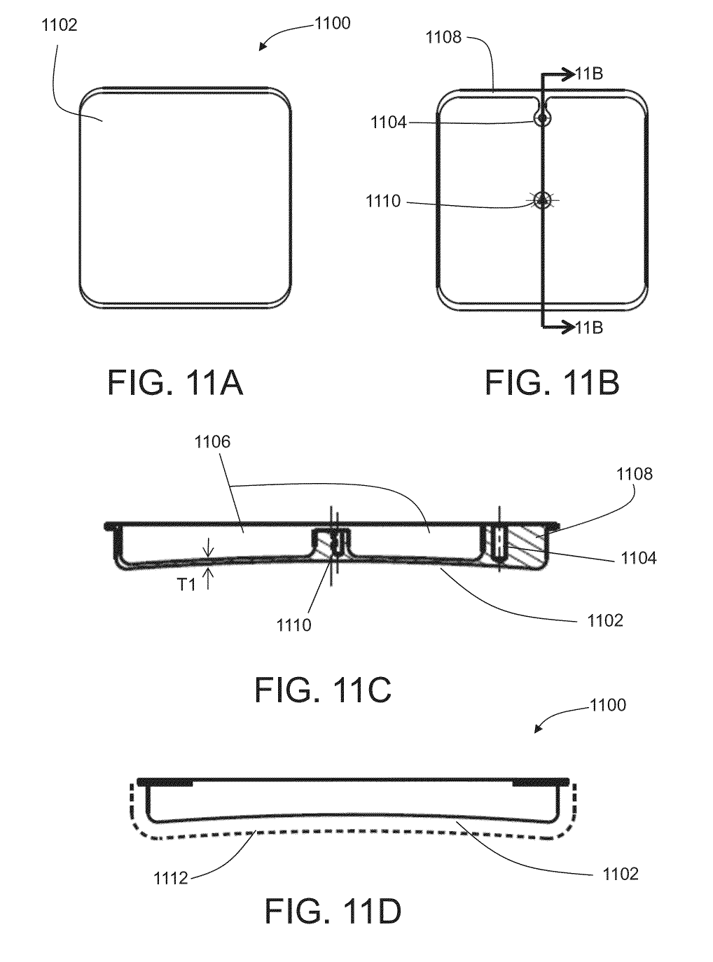

Another variation of a surface-contoured electrode that may be used with a RF system is depicted in FIGS. 11A-11D. While the surface-contoured electrode of FIGS. 3A-3C comprises a tissue-contacting surface that protrudes with a substantially convex geometry, the surface-contoured electrode of FIGS. 11A-D comprises a tissue-contacting surface that is recessed, having a substantially concave geometry. Electrodes with concave or convex surface contour geometries may provide more consistent contact with convex or concave anatomical features, respectively. Uniform electrode contact with the tissue may allow for the even distribution of current and/or heat across that tissue. For example, electrodes with a convex surface contour may be useful for applying RF energy to recessed tissue regions, such as upper flank anatomy between sternum and pelvis or the area near the obliques while electrodes with a concave surface contour may be useful for applying RF energy to protruded tissue regions, such as the abdomen. An electrode may be substantially flat. Concave and/or convex surface contours may also help dissipate heat that may result from activating the electrode. Alternatively or additionally, an electrode may have regions that are concave or convex, and other regions that are flat. As described previously, the shape of an electrode may be any desired shape, for example, circular (as depicted in FIGS. 3A-3C) or square (as depicted in FIGS. 11A-11D).

FIG. 11A is a bottom view of a square concave electrode 1100 depicting the tissue-contacting surface 1102. FIG. 11B is a top view of the electrode 1100 where there may be a hole 1104 for attaching the electrode 1100 to a handpiece. For example, the hole 1104 may be configured to receive an electrically conductive conduit that connects the electrode an RF energy source. For example, the hole 1104 may be configured to receive a screw that is connected to an RF energy source (e.g., as described and depicted in FIGS. 10C and 10D). FIG. 11C depicts a cross-section of electrode 1100 along the line 11B-11B. As shown there, a central portion 1106 of the electrode 1100 may have a cavity, with solid portions along an edge 1108. Any of the electrodes disclosed herein may be a solid electrode made of aluminum or any other suitable conductive material such that the electric potential is equal across the entire electrode (i.e., equipotential electrode). The thickness T1 of the electrode 1100 in the hollow central portion 1106 may be from about 0.25 mm to about 1.25 mm, e.g., 0.63 mm. The thickness of the electrode may be adjusted and selected to distribute the heat generated by the electrode during the application of RF energy. The electrode 1100 may also comprise one or more temperature sensors (thermistors) within the cavity 1106 of the electrode for sensing the temperature of the skin. For example, a temperature sensor may be located in the center of the electrode, within a hole 1110. The temperature sensor may be in communication with a controller to provide temperature feedback to modulate the voltage and/or current applied to the electrode. The tissue-contacting portion 1102 may be concave, having a radius of curvature between about −400 mm to about +400 mm. As shown in FIG. 11D, the electrode 1100 may comprise one or more dielectric coatings or films 1112 disposed over its tissue contacting surface. The film 1112 may be any of the films previously described, and applied over the electrode 1100 using similar methods.

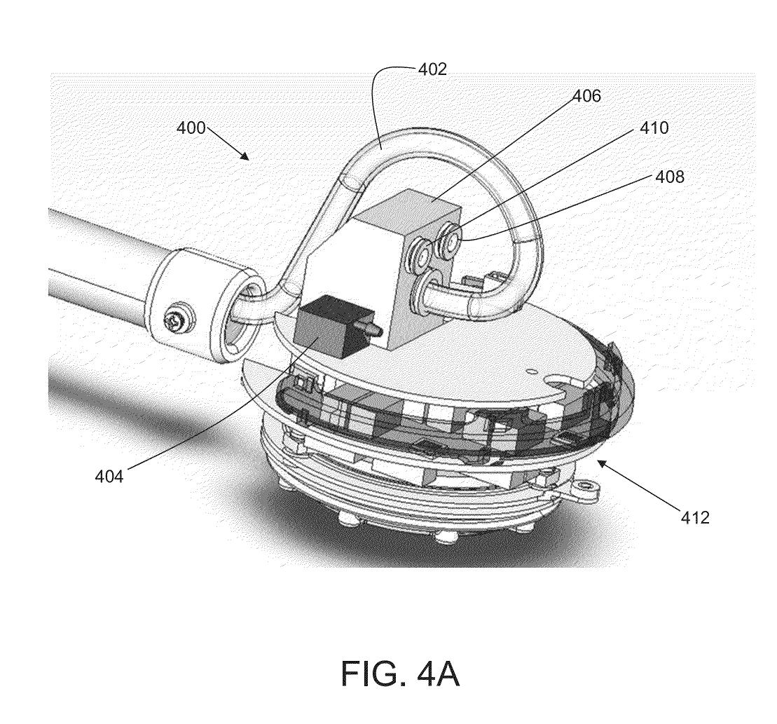

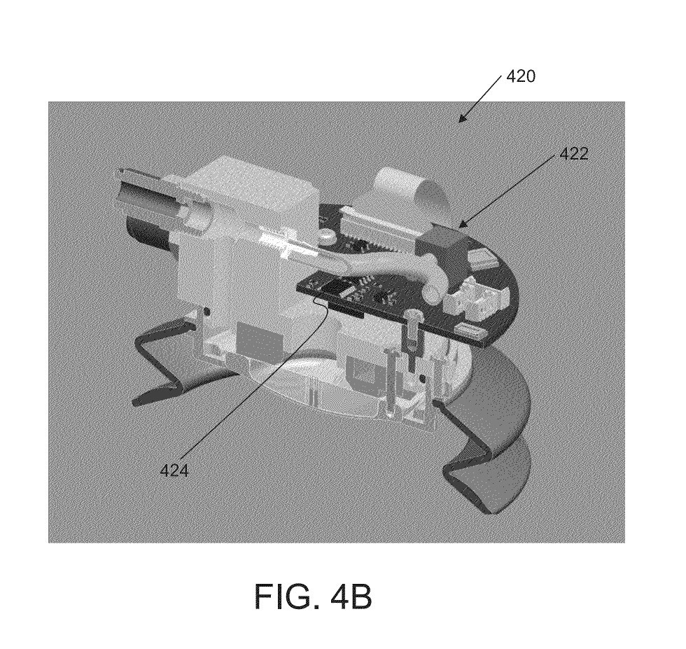

In addition to having one or more surface-contoured electrodes, a handpiece of a RF system for heating subcutaneous tissue may comprise electrical and mechanical components to support optional functions, such as the application of vacuum to a tissue region. Some handpieces may optionally comprise a heat dissipation system to help ensure that the electrode and its associated electrical circuitry do not overheat in the course of patient treatment. FIG. 4A depicts an example of a handpiece 400 (with the housing and bellows removed for the sake of clarity) comprising a vacuum system and a heat dissipation system. The vacuum system may comprise a vacuum tube 402 and a pressure sensor 404. The pressure sensor 404 may be configured to measure the amount of pressure (e.g., negative pressure) applied to a patient's skin and convey that information to the system console and optionally displayed to the practitioner operating the system. In some variations, the pressure sensor 404 may provide a feedback signal to the system controller, which may then regulate the timing and magnitude of the vacuum source within a predetermined range. The vacuum tube 402 may be in fluid connection with the one or more apertures of the bellows frame to create a vacuum in the bellows chamber. A heat dissipation system may comprise a fluid distribution hub 406 with a fluid inlet 408 and a fluid outlet 410, and one or more tubes to connect the inlet and outlet with a fluid reservoir (not shown). The fluid distribution hub 406 may be in fluid communication with one or more conduits within the handpiece 400 to transport fluid to areas of the handpiece that may increase in temperature during use. For example, the distribution hub 406 may be connected to conduits that are in close proximity to the electrode (e.g., the conduits may be in a plate that is located adjacent to the electrode), which may help prevent the electrode from heating beyond a threshold temperature. The conduits may be connected via one or more tube to the outlet 410 to transport any excess heat away from the handpiece. While a fluid-based heat dissipation system is described and depicted, other types of heat dissipation systems and heat sinks may be used (e.g., fans, heat dissipation structures made of heat conductive materials, and the like).

In some variations, a handpiece may comprise one or more filtering components to help reduce electrical noise. For example, handpiece 400 may comprise filtering layer 412, as shown in FIG. 4A. While electrical noise filters may be located on a separate printed circuit board and/or layer, such filters may be located on a single PCB board or layer, such as is depicted in FIG. 4B. As illustrated there, handpiece 420 comprises a single printed circuit board layer that serves as a substrate for the vacuum system 422 and other electrical components (e.g., electrical noise filters, sensors, control circuitry, etc.). Handpieces may have one, two, three or more printed circuit board layers, as may be suitable for attaining the desired amount of electrical isolation and handpiece size.

### Methods

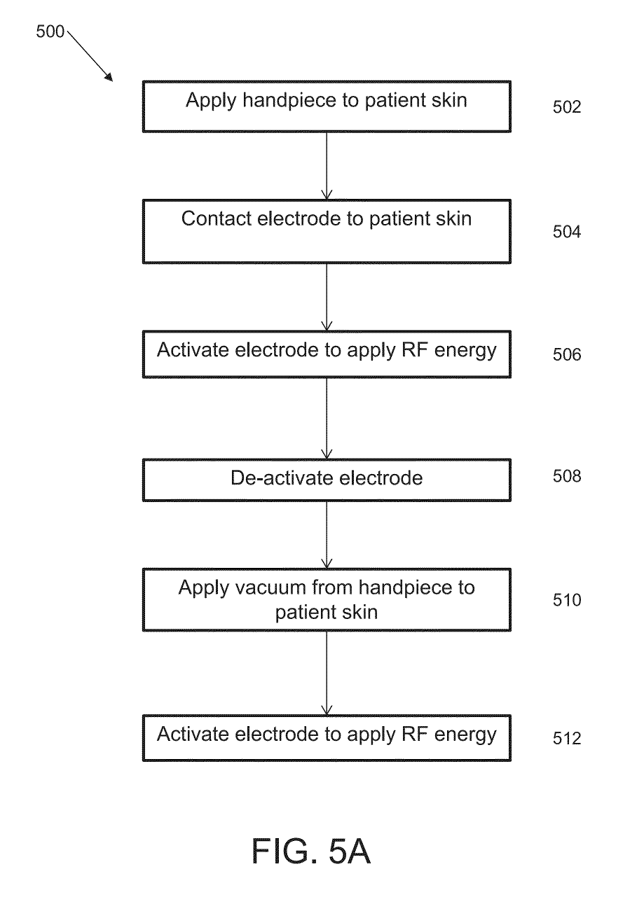

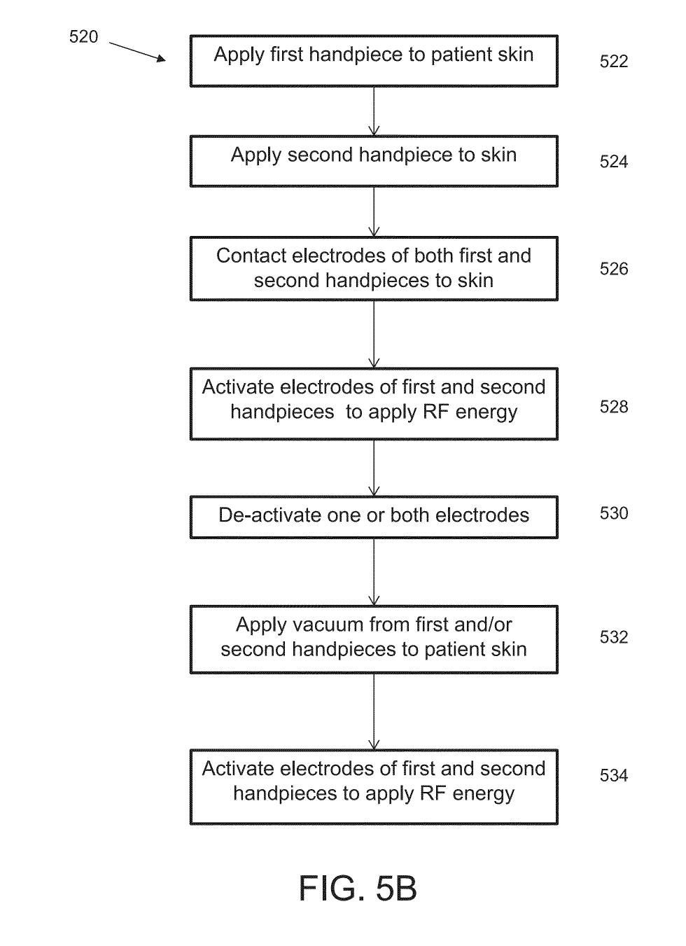

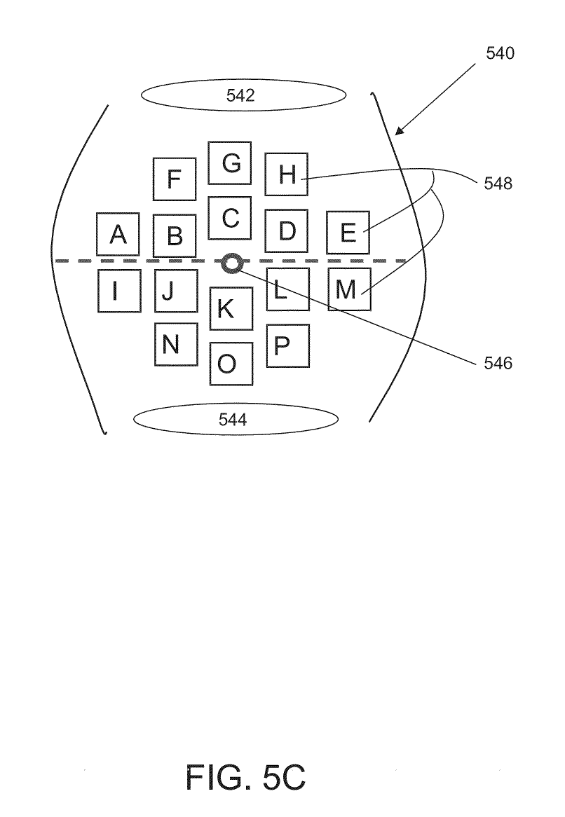

Variations of methods for heating subcutaneous adipose tissue using the systems described above are depicted in FIGS. 5A-5C. Method 500 may be used with an RF system having one handpiece or two handpieces, where the second handpiece is not activated. The method 500 may comprise applying the handpiece to a patient's skin 502, contacting the electrode the patient's skin 504 (which may be confirmed by checking a signal from the one or more skin contact sensors in the handpiece), and activating the electrode and supplying RF energy to the electrode 506. A ground pad may also be applied to the patient at a non-targeted region of the body prior to activating the electrode. During the application of RF energy to the electrode, the temperature of the skin may be monitored by the thermistors in the electrode, and/or via patient feedback. At a certain point, either based on temperature readings and/or patient feedback, the electrode may be de-activated 508. Optionally, vacuum may be applied to the patient's skin 510, which may help increase blood perfusion to the area of treatment. Increased blood perfusion may help to dissipate any excess heat that may have accumulated in the target region. After a sufficient time period (which may be indicated by either the patient, temperature feedback, and/or a pre-programmed time interval), the electrode may be re-activated to apply RF energy to the target region 512. Steps 506 to 512 may be repeated across the same or different regions of the patient, depending on the treatment plan devised by the practitioner. In some variations, vacuum may be applied simultaneously with RF energy application.

FIG. 5B depicts another variations of a method that uses two handpieces where each handpiece has one electrode. The method 520 may comprise applying the first handpiece to a patient's skin 522, applying the second handpiece to different region of the patient's skin 524, contacting the electrodes of both handpieces to the patient's skin 526 (which may be confirmed by checking a signal from the one or more skin contact sensors in the handpiece, and/or by detecting changes in the load impedance of the electrodes), and activating the electrodes of both handpieces and supplying RF energy to the electrodes 528. A ground pad may also be applied to the patient at a non-targeted region of the body prior to activating the electrodes of the first and second handpieces. During the application of RF energy to the electrodes, the temperature of the skin may be monitored by the thermistors in the electrodes, and/or via patient feedback. At a certain point, either based on temperature readings and/or patient feedback, the electrodes of one or both handpieces may be de-activated 530. Optionally, vacuum may be applied to the patient's skin from one or both handpieces 532, which may help increase blood perfusion to the area of treatment. After a sufficient time period (which may be indicated by either the patient, temperature feedback, and/or a pre-programmed time interval), the electrodes of one or both handpieces may be re-activated to apply RF energy to the target region 534. Steps 528 to 534 may be repeated across the same or different regions of the patient, depending on the treatment plan devised by the practitioner.

FIG. 5C schematically depicts the placement of a handpiece during the treatment of an abdominal body region, for example. A treatment region may be divided into a plurality of treatment zones 548. For example, there may be 1, 2, 3, 4, 5, 8, 10, 12, 15, 20, 25 or more treatment zones depending on the size and shape of the body region to be treated. For example, an abdominal region may have 14 to 20 zones, a thigh region may have 6 to 10 zones, and a flank region may have 6 to 10 zones. A treatment region located in the abdomen may comprise the portion of the abdomen 520 bounded by the rib cage 542 and the pelvis 544. As shown in FIG. 5C, there may be sixteen treatment zones 548 arranged around the navel 546. The treatment zones 548 may be symmetrically or asymmetrically arranged around the navel 546 (e.g., may be bilaterally symmetric with respect to a line that intersects the navel 546). Any one of the previously described handpieces may be placed at a given treatment zone for a certain period of time at a certain power or current level or vacuum before it is moved to another treatment zone. In some variations, a handpiece may be placed at a treatment zone 548 for about 10 seconds, 20, seconds, 30 seconds, 60 seconds, 150 seconds, e.g., 120 seconds, before it is moved to another treatment zone. For example, a handpiece may be placed at treatment zone A for 120 seconds, then moved to treatment zone B for 120 seconds, then moved to treatment zone C for 120 seconds, and so on, until all the treatment zones A to P have been contacted once. Optionally, after treatment zone P has been treated, the handpiece may positioned back at treatment zone A, then treatment zone B, etc. such that all the treatment zones are treated a second time. For a dual-handpiece RF system, two treatment zones 548 may be treated simultaneously, which may attain a bipolar and/or a monopolar treatment effect (depending on the phase shift between the two handpieces, as well as the location of a ground pad). The use of two handpieces may also help expedite the treatment session. For example, a first handpiece may be placed at treatment zone A, a second handpiece may be placed at treatment zone I, and then both handpieces may be activated. After some time (e.g., about 10 seconds, 20, seconds, 30 seconds, 60 seconds, 120 seconds, 150 seconds), both handpieces may be removed from treatment zones A and I, and then moved to treatment zones B and J for the desired time period, then treatment zones C and K, etc. A handpiece may provide vacuum suction to a treatment zone 548 before it is moved to another treatment zone (e.g., similar to the methods described in FIGS. 5A and 5B).

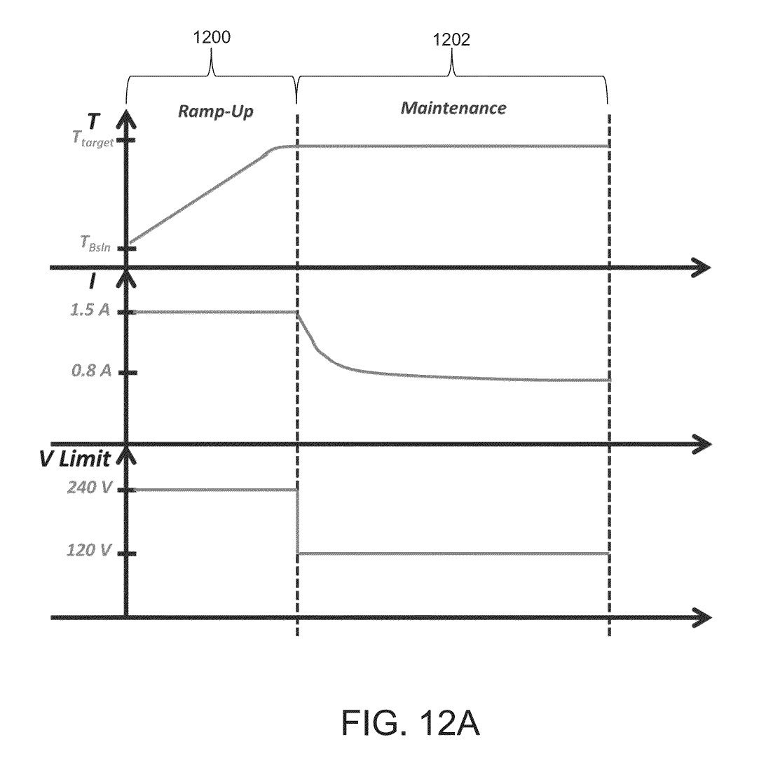

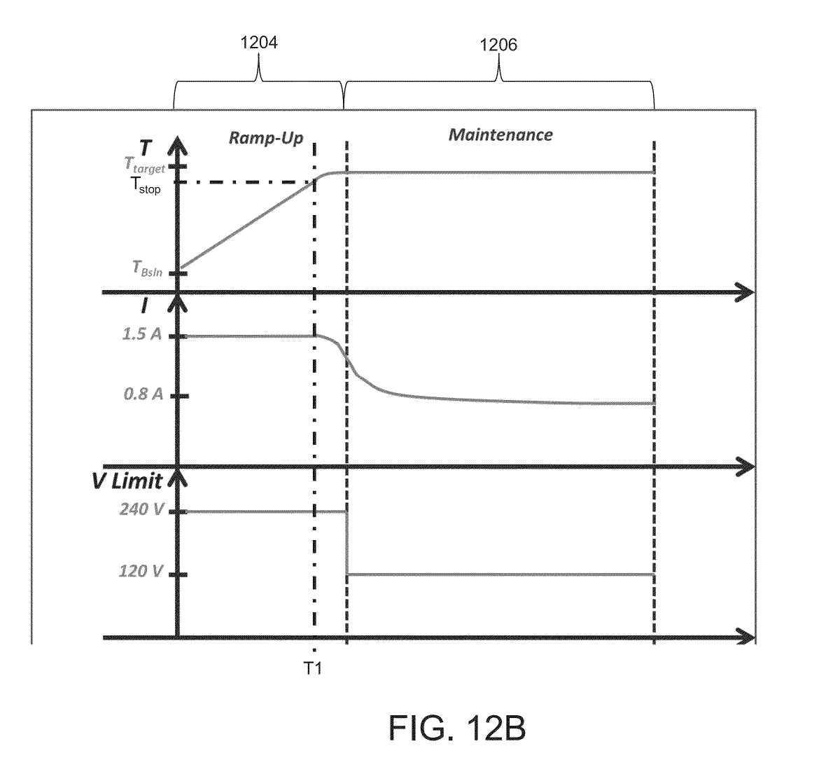

In some methods, the voltage applied to the electrodes for heating subcutaneous adipose tissue may vary depending on the temperature of the electrode and/or the skin. For example, when a handpiece comprising an electrode is contacted to a patient's skin, the voltage applied to the electrode may be increased until the temperature of the electrode and/or skin is at a predetermined threshold temperature. Once the temperature of the electrode and/or skin attains the predetermined target temperature, the voltage applied to the electrode may be reduced to maintain a desired temperature. FIG. 12A depicts voltage and current waveforms that drive the electrode to attain and maintain a certain tissue and/or electrode temperature. The voltage and current levels applied to the electrode may be modulated based on temperature data from a temperature sensor in the electrode. In a first ramp-up phase 1200, the voltage applied to the electrode may be about 240 V, and/or may be limited to about 240 V. The current flowing to the electrode may be about 1.5 A. The target temperature of the tissue and/or electrode may be from about 43 degrees Celsius to about 52 degrees Celsius. The ramp-up phase 1200 may be about 45 seconds to about 90 seconds, e.g., about 60 seconds, depending upon the length of time it takes for the tissue and/or electrode to attain the target temperature. After the target temperature is reached (based on temperature data fed back from the temperature sensor), the current and voltage supplied to the electrode may be reduced so that the temperature of the tissue and/or electrode is not further increased. In the maintenance phase 1202, the voltage applied to the electrode may be adjusted in accordance with the sensed electrode and/or tissue temperature, but may be limited to no more than about 120 V. In some variations, the voltage applied to the electrode is about 120 V. The current to the electrode may be about 0.2 A to about 1 A, e.g., about 0.7 A or about 0.8 A. For example, the voltage applied to the electrode may be adjusted such that the electrode and/or tissue temperature is maintained at the target temperature (e.g., reduced if the sensed temperature exceeds the target temperature and increased if the sensed temperature is less than the target temperature). The maintenance phase 1202 may be about 180 seconds. In some variations, the length of the maintenance phase 1202 may depend on the target temperature. For example, the maintenance phase may be about 60 seconds if the tissue reaches a target temperature of about 50 degrees Celsius, which may result in the death of 45% of the adipose tissue cells in contact and/or in close proximity with the electrode. Alternatively or additionally, the maintenance phase may be about 180 seconds if the tissue reaches a target temperature of about 45 degrees Celsius, which may result in the death of 60% of the adipose tissue cells in contact and/or in close proximity with the electrode. In the maintenance phase, the maximum voltage output of the controller to the electrode may be limited to about 120 V, which may help prevent voltage spikes as the electrode is lifted from the skin (e.g., help to prevent plasma arc formation between the skin and the electrode as a result of sweating).

FIG. 12B depicts another variation of voltage and current waveforms that may be applied to an electrode to heat and maintain tissue at a particular target temperature. During the ramp-up phase 1204, the voltage applied to the electrode may be about 240 V, and may drive a current of about 1.5 A. Once the sensed temperature reaches a temperature Tₛₜₒₚ (where Tₛₜₒₚ is less than T_{target}), the amount of current may be reduced at timepoint T1. The current value is adjusted by regulating the voltage. This may help to ensure that the tissue is not heated beyond the target temperature, which may help prevent discomfort to the patient. The voltage and current applied to the electrode during the maintenance phase 1206 may be similar to the maintenance phase described above. It should be noted that the voltage values discussed above relate to single handpiece systems. It is possible to operate with two handpieces and then the voltages would be scaled up accordingly.

### EXAMPLES

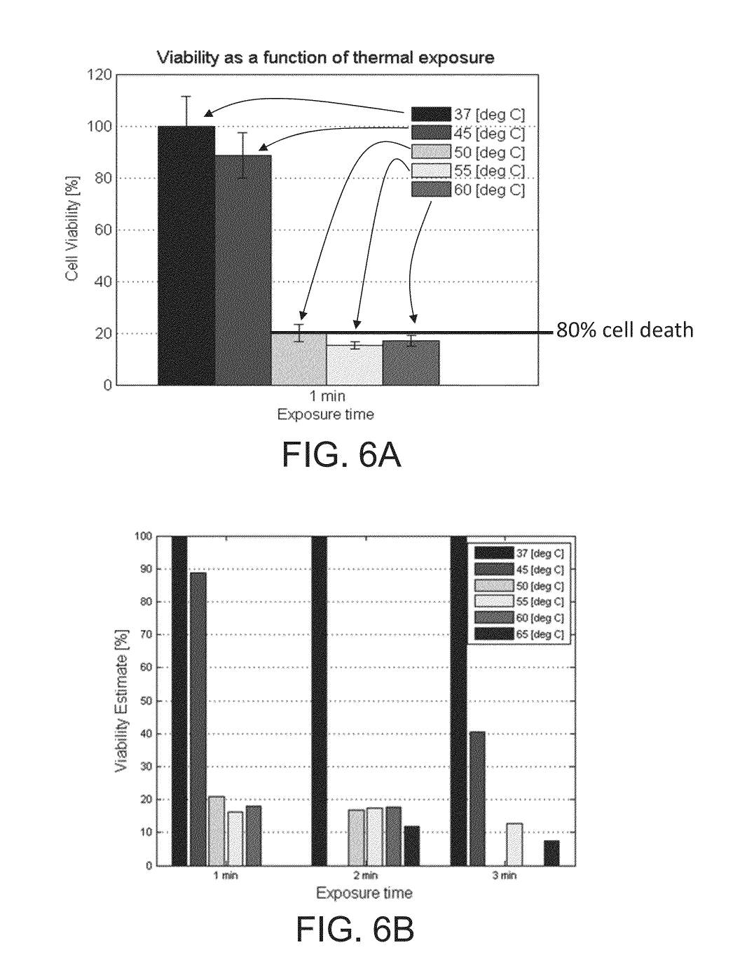

FIGS. 6A and 6B are histograms that plot the results of experiments to determine the relationship between temperature and human adipose cell viability. Human adipocyte cells were cultured in six wells, where the cells in each well were exposed to various temperatures ranging from 37° C. to 65° C. for one, two, and three minutes. The percent of adipocyte cells that remained viable 72 hours after heat exposure are reflected in the histogram shown in FIG. 6A. Eighty percent of the adipose cells exposed to temperatures of 50° C. for one minute were no longer viable after 72 hours. Also, 60% of adipocytes exposed to temperatures of 45° C. for 3 minutes were no longer viable after 72 hours, as shown in FIG. 6B.

Additional experiments and description regarding heating of adipose tissue using an RF applicator with concentric rings may be found in several papers, including “Controlled volumetric heating of subcutaneous adipose tissue using a novel radiofrequency technology” (by W. Franco, A. Kothare, D. J. Goldberg published in Lasers in Surgery and Medicine 41:745-750, 2009) and “Hypothermic injury to adipocyte cells by selective heating of subcutaneous fat with a novel radiofrequency device: feasibility studies” (by W. Franco, A. Kothare, S. J. Ronan, R. C. Grekin, and T. H. McCalmont published in Lasers in Surgery and Medicine 42:361-370, 2010), each of which is hereby incorporated by reference in its entirety. Concentric electrodes for tissue heating are also described in U.S. Pat. Publ. No. 2008/0312651 titled “Apparatus and Methods for Selective Heating of Tissue” and U.S. Pat. Publ. No. 2009/0171341 titled “Dispersive Return Electrode and Methods”, and spiral electrodes are disclosed in U.S. Pat. Publ. No. 2009/0171346 titled “High Conductivity Inductively Equalized Electrodes and Methods”, each of which is hereby incorporated by reference in its entirety.

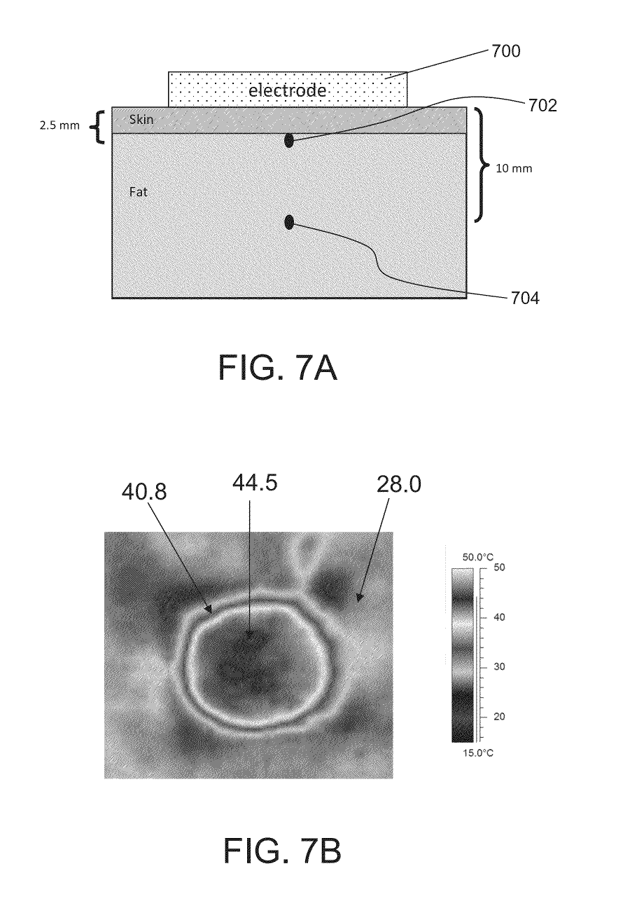

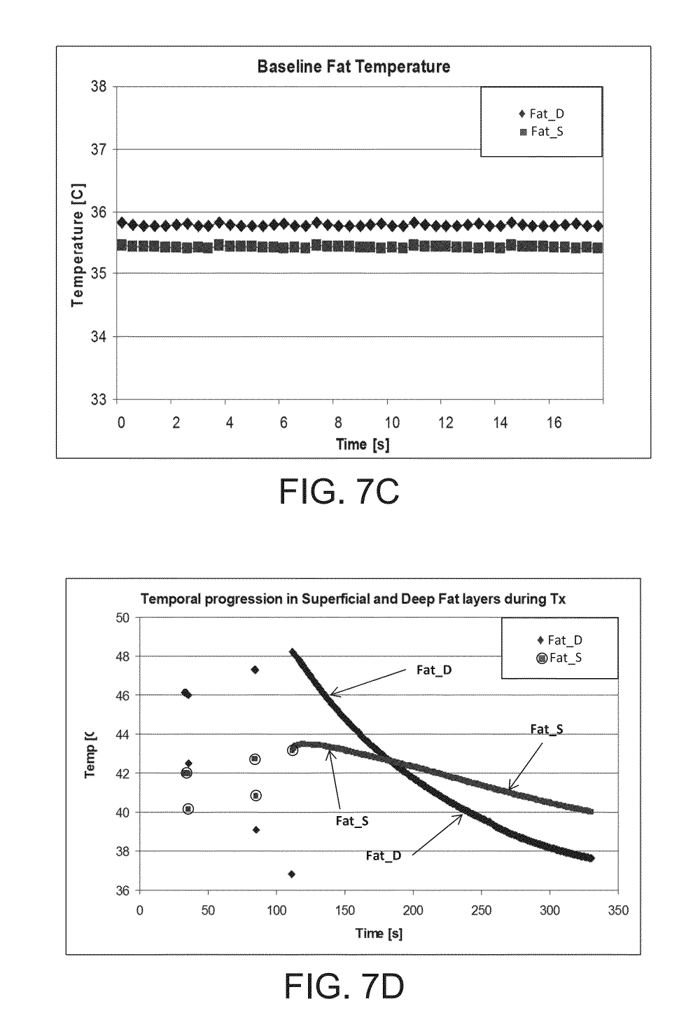

FIGS. 7A and 7B depict an experiment to assess how the heat distribution varies at different tissue depths when an electrode is placed on the surface of the tissue. FIG. 7A schematically depicts the experimental set up, where a surface-contoured (e.g., domed) electrode 700 is placed on the surface of a porcine tissue specimen, a first thermocouple probe 702 is positioned 2.5 mm beneath the surface of the skin, and a second thermocouple probe 704 is positioned 10 mm beneath the surface of the skin. No active cooling is applied to the skin or the electrode during the course of the experiment. FIG. 7B is an image taking with an infrared camera that depicts the temperature of the skin surface immediately after RF exposure from the electrode 700. As shown there, the outer circumference of the domed electrode 700 is at a temperature of about 40.8° C., the inner portion of the domed electrode is at a temperature of about 44.5° C., and the tissue surrounding the electrode (e.g., outside of the circumference of the electrode) is at a temperature of about 28.0° C. FIG. 7C is a graph depicting that tissue that is located 10 mm beneath the skin surface (“Fat_D” indicated by diamond-shaped points) is at a temperature that is elevated above the temperature of tissue located 2.5 mm beneath the surface of the skin (“Fat_S” indicated by square-shaped points). FIG. 7D is a graph depicting the change in temperature over time for tissue 10 mm beneath the surface of the skin (“Fat_D” indicated by diamond-shaped points) and for tissue located 2.5 mm beneath the surface of the skin (“Fat_S” indicated by circled square-shaped points). These experiments indicate a degree of uniform diffuse heating beneath the skin.

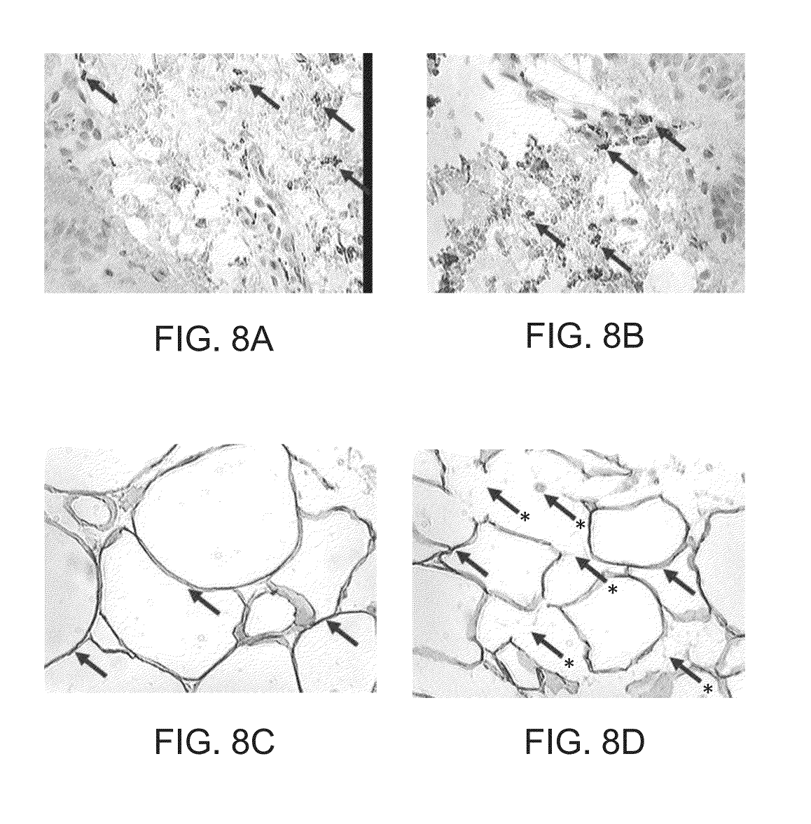

FIGS. 8A-8D depict an experiment to demonstrate changes in macrophage activity as well metabolic activity in human adipose cells due to heat treatment by applying RF energy using a domed electrode. FIG. 8A depicts macrophage activity in fat tissue stained with CD-68 before the application of RF energy with the domed electrode, where macrophages seen as well-defined cell walls indicated by the arrows. FIG. 8B depicts macrophage activity in fat tissue stained with CD-68 ten weeks after the application of RF energy with the domed electrode. FIG. 8C depicts fat cells stained with perilipin before the application of RF energy with the domed electrode, where areas of normal basal metabolic activity are indicated by the arrows. FIG. 8D depicts fat cells stained with perilipin ten weeks after the application of RF energy with the domed electrode, where areas of increased metabolic activity are indicated by arrows with asterisks and areas of normal basal metabolic activity are indicated by arrows without asterisks. Increase in overall metabolic activity is seen by the smaller size of the fat cells in FIG. 8D.

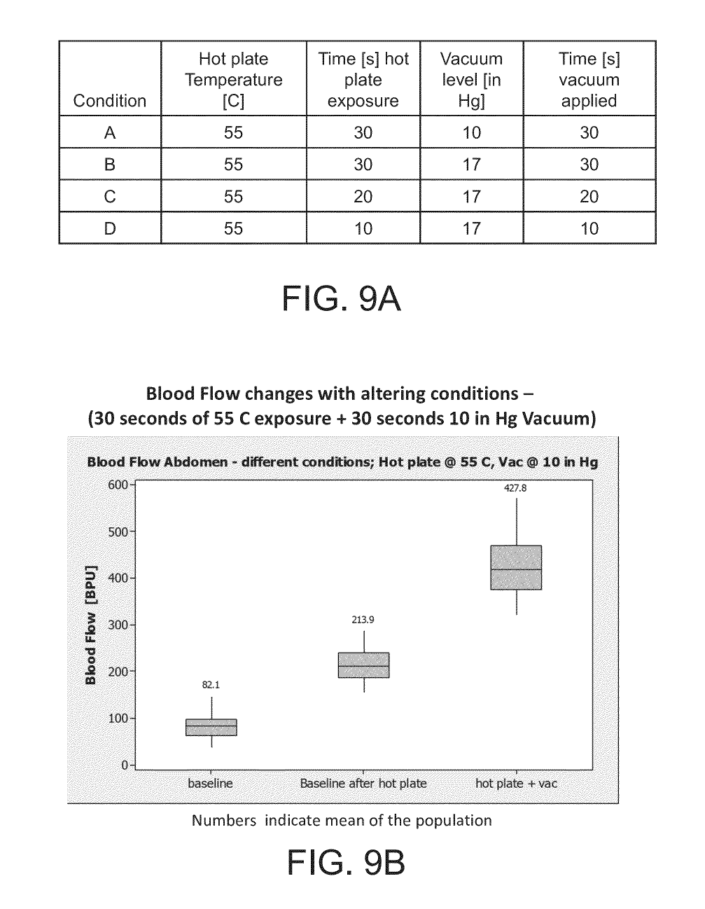

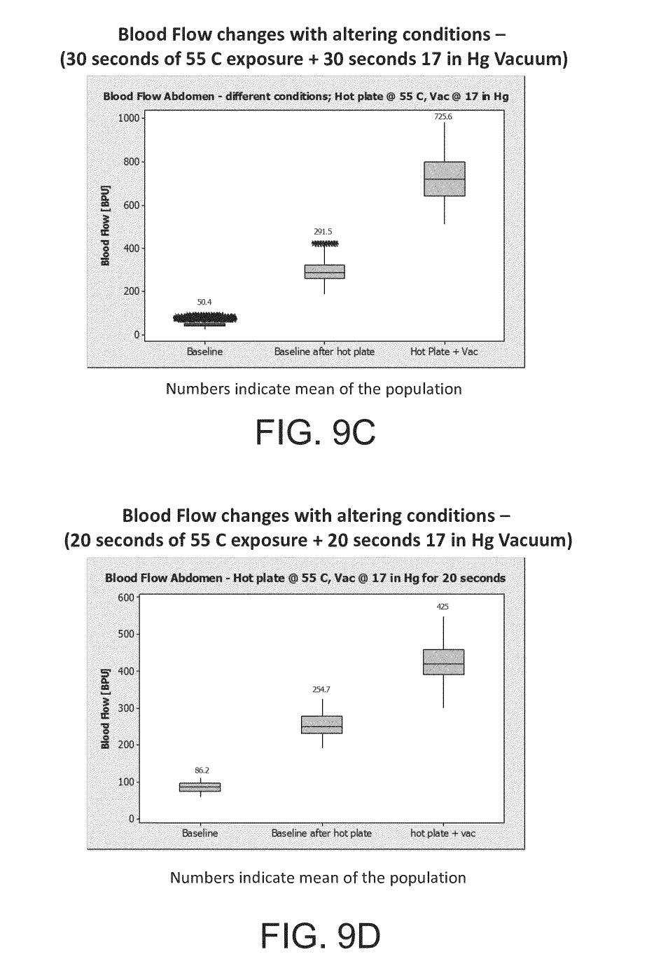

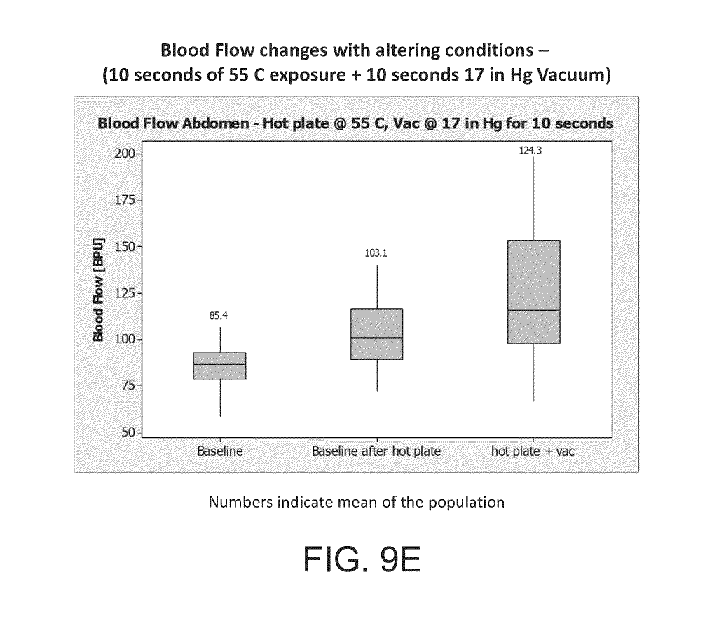

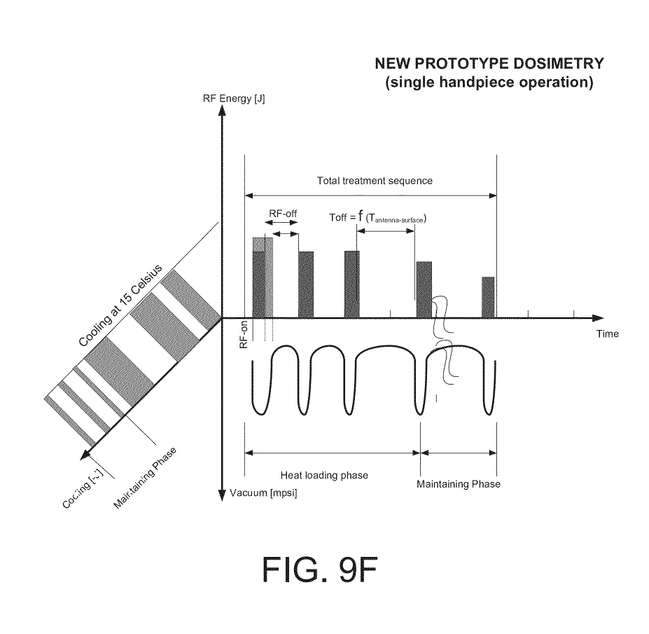

FIGS. 9A-9G depict a series of experiments and plots that evaluate pressure-induced vasodilation after heating in humans. FIG. 9A is a chart showing the various experimental conditions applied to the right abdomen of a test subject. Heat was applied to the subject with a handpiece with a hot plate. Resting blood perfusion levels were measured at the test tissue region, then a hot plate set to 55° C. was moved over the test tissue region for the specified time period, after which the blood perfusion in the test area was measured again. Next, vacuum was applied over the test tissue region for the specified time period, and after the vacuum was released, the blood perfusion was measured again. FIG. 9B is a graph that shows the changes in blood flow after exposing the test tissue region to a 55° C. hot plate for 30 seconds, and then exposing the test tissue region to 10 in Hg for 30 seconds. FIG. 9C is a graph that shows the changes in blood flow after exposing the test tissue region to a 55° C. hot plate for 30 seconds, and then exposing the test tissue region to 17 in Hg for 30 seconds. FIG. 9D is a graph that shows the changes in blood flow after exposing the test tissue region to a 55° C. hot plate for 20 seconds, and then exposing the test tissue region to 17 in Hg for 20 seconds. FIG. 9E is a graph that shows the changes in blood flow after exposing the test tissue region to a 55° C. hot plate for 10 seconds, and then exposing the test tissue region to 17 in Hg for 10 seconds. These results are consistent with the findings in other studies, such as the study described in “Using wavelet analysis to characterize the thermoregulatory mechanisms of sacral skin blood flow”, Mary Jo Geyer, PhD, PT; Yih-Kuen Jan, PhD, PT; David M. Brienza, PhD; Michael L. Boninger, MD, Journal of Rehabilitation Research & Development. FIG. 9F is a timing diagram that demonstrates the application of RF energy and vacuum suction to a tissue region using a single handpiece, where the vacuum suction is increased when the RF energy is turned off, and the vacuum suction is decreased when the RF energy is turned on (e.g., where the RF energy and vacuum are applied to a tissue region out of phase from each other). The time intervals between vacuum pulses and/or RF pulses may be varied to adjust the temperature of a tissue region by increasing blood perfusion (by increasing the vacuum suction and decreasing RF energy) if the tissue is too hot, and decreasing blood perfusion (by decreasing the vacuum suction and increasing RF energy) if the tissue is too cold.

What is claimed is:

1. A system for heating subcutaneous tissue comprising:
a handpiece having an electrode, wherein the electrode has a tissue-contacting surface and a coating disposed over the tissue-contacting surface; and
a controller configured to apply a voltage to the electrode to apply RF energy to tissue.

2. The system of claim 1, wherein the coating comprises a material with dielectric properties.

3. The system of claim 2, wherein the coating has a thickness from about 3 micrometers to about 100 micrometers.

4. The system of claim 3, wherein the coating comprises polyurethane.

5. The system of claim 1, wherein the tissue-contacting surface of the electrode is convex.

6. The system of claim 1, further comprising a temperature sensor that is located within the electrode and in contact with the tissue-contacting surface.

7. A system for heating subcutaneous tissue comprising:
a handpiece having an electrode, wherein the electrode has a tissue-contacting surface and a dielectric film disposed over the tissue-contacting surface;
a temperature sensor in contact with the tissue-contacting surface of the electrode; and
a controller coupled to the temperature sensor and configured to apply a voltage to the electrode to apply RF energy to the tissue, wherein the controller is programmed to apply a first voltage level during a first phase while the tissue temperature is being increased and wherein during a second phase following the first phase, the voltage is adjusted to maintain a target temperature of the tissue and wherein during the second phase, the voltage level is restricted not to exceed a second voltage level, said second voltage level being less than the first voltage level.

8. A system as recited in claim 7 wherein during the first phase, the first voltage level is maintained until the target temperature is reached.

9. A system as recited in claim 7 wherein the first voltage level is maintained during the first phase until a stop temperature of the tissue is reached, said stop temperature being below the target temperature.

10. A device for heating subcutaneous tissue comprising:
a handpiece having an electrode, wherein the electrode has a tissue-contacting surface and a dielectric film disposed over the tissue-contacting surface, wherein the film is uniformly stretched over the tissue-contacting surface.

11. The device of claim 10, wherein the dielectric film is a first dielectric film, and the electrode has a second dielectric film that is uniformly stretched over the tissue-contacting surface and on top of the first dielectric film.

12. The device of claim 11, wherein the total thickness of the first and second films is 50 micrometers.

13. The device of claim 11, wherein each of the first and second films is stretched over the tissue-contacting surface by attaching the film to a frame, applying an adhesive to one side of the film, and applying the frame to the electrode at an angle such that the side of the film with the adhesive contacts the electrode without any air pockets between the tissue-contacting surface and the film.

14. The device of claim 13, wherein the adhesive is an acetate adhesive.

15. The device of claim 11, wherein the first and the second films are polyurethane films.

16. The device of claim 10, wherein the film has an electrical conductivity range of about 1×10⁻⁸ S/m to about 100×10⁻⁸ S/m.

17. The device of claim 10, wherein the film has a permittivity range from about 1 to about 7.

18. The system of claim 10, wherein the film has a thickness from about 10 micrometers to about 100 micrometers.

19. The system of claim 10, wherein tissue-contacting surface has a radius of curvature from about −400 mm to about +400 mm.

20. The system of claim 10, wherein the tissue-contacting surface is flat.

21. The system of claim 19, wherein the tissue-contacting surface is concave.

22. The system of claim 19, wherein the tissue-contacting surface is convex.

23. The system of claim 10 wherein said electrode is at least 500 microns thick.
